(19) United States
(12) Patent Application Publication
(10) Pub. No.: US 2020/0179273 A1
(43) Pub. Date: Jun. 11, 2020

(54) LONG-ACTION IMPLANT FOR TREATMENT OF INFECTIOUS DISEASES

(71) Applicant: Merck Sharp & Dohme Corp., Rahway, NJ (US)

(72) Inventors: Stephanie Elizabeth Barrett, Cranford, NJ (US); Seth P. Forster, Fort Washington, PA (US); Marian E. Gindy, North Wales, PA (US); Jay A. Grobler, Gwynedd, PA (US); David Brian Olsen, Lansdale, PA (US); Ryan Stevenson Teller, Sierra Madre, CA (US)

(73) Assignee: Merck Sharp & DohmeCorp, Rahway, NJ (US)

(21) Appl. No.: 16/614,855

(22) PCT Filed: Jun. 1, 2018
(86) PCT No.: PCT/US18/35520

§ 371 Date: Nov. 19, 2019

Related U.S. Application Data
(60) Provisional application No. 62/515,582, filed on Jun. 6, 2017.

(51) Int. Cl. A61K 9/00; A61K 31/47; A61K 31/438; A61K 31/497; A61K 31/4409; A61K 47/36; A61K 47/12; A61K 47/26

(52) U.S. Cl. CPC A61K 9/0024; A61K 31/47; A61K 31/438; A61K 47/26; A61K 31/4409; A61K 47/36; A61K 47/12; A61K 31/497

(57) ABSTRACT

The present invention relates to long-acting implants useful for the treatment of bacterial infections, particularly mycobacterial infections. The invention also relates to methods of use of such long-acting implants for the treatment of mycobacterial infections such as those caused by Mycobacteria tuberculosis.

### FIELD OF THE INVENTION

The present invention relates to long-acting implants useful for the treatment of bacterial infections, particularly mycobacterial infections. The invention also relates to methods of use of such long-acting implants for the treatment of mycobacterial infections such as those caused by Mycobacteria tuberculosis.

### BACKGROUND OF THE INVENTION

Mycobacterium is a genus of bacterium, neither truly gram-positive nor truly gram-negative, and includes pathogens responsible for tuberculosis (M. tuberculosis) and leprosy (M. leprae). Tuberculosis (TB), in particular, is considered to be one of the world's deadliest diseases. According to World Health Organization, in 2015, there were an estimated 10.4 million new (incident) TB cases worldwide, of which 5.9 million (56%) were among men, 3.5 million (34%) among women and 1.0 million (10%) among children. People living with HIV accounted for 1.2 million (11%) of all new TB cases. See, Global tuberculosis report 2016 published by the World Health Organization. There were an estimated 1.4 million TB deaths in 2015, and an additional 0.4 million deaths resulting from TB disease among people living with HIV. See, Global Tuberculosis Report 2016 published by the World Health Organization.

Available oral treatment regimens to cure or prevent tuberculosis infection, such as isoniazide and rifampin, are complex and long, typically requiring daily doses for 6 to 9 months to treat active TB infections. See, Global Tuberculosis Report 2016 published by the World Health Organization. This can lead to treatment fatigue and patients not completing the prescribed dosing regimen.

Moreover, there are approximately 56 million people with latent TB infection (LTBI). Individuals with LTBI harbor Mycobacterium tuberculosis that may progress to active TB at some point in their lives. See, Treatment of Latent Tuberculosis Infection; Haley, Connie; Microbiol Spectr. 2017 April. Modeling studies suggest that if only 8% of these individuals with LTBI were treated annually, overall global incidence would be 14-fold lower by 2050 compared to incidence in 2013.

TB and LTBI presents a unique opportunity to develop a long-acting, drug eluting implant capable of achieving sufficient drug pharmacokinetics over a period of weeks or months after a single administration.

### SUMMARY OF THE INVENTION

The present invention relates to long-acting implants useful for the treatment of bacterial infections, particularly mycobacterial infections. The invention also relates to methods of use of such long-acting implants for the treatment of mycobacterial infections such as those caused by Mycobacteria tuberculosis.

In certain embodiments the implants described herein comprise at least one anti-mycobacterial compound; and nonpolymer pharmaceutical excipients, wherein at least one nonpolymer pharmaceutical excipient is a bulking agent.

Also described herein are implants comprising at least one anti-mycobacterial compound; and pharmaceutical excipients, wherein at least one pharmaceutical excipient is a bulking agent.

In certain embodiments, the implants described herein can be adapted for subdermal implantation.

In certain embodiments, the implants described herein comprise an anti-mycobacterial compound, wherein the anti-mycobacterial compound is an anti-tuberculosis compound.

In certain embodiments, the implants described herein comprise an anti-mycobacterial compound, wherein the anti-mycobacterial compound has low aqueous solubility.

In certain embodiments, the implants described herein comprise an anti-mycobacterial compound, wherein the anti-mycobacterial compound has an aqueous solubility below 200 μg/ml.

In certain embodiments, the implants described herein comprise an anti-mycobacterial compound, wherein the anti-mycobacterial compound is bedaquiline, delamanid, linezolid, PBTZ169, pretomanid, Q203, rifampicin, rifabutin, rifapentine, isoniazid or sutezolid.

In certain embodiments, the implants described herein comprise an anti-mycobacterial compound, wherein the anti-mycobacterial compound is bedaquiline.

In certain embodiments, the implants described herein comprise an anti-mycobacterial compound, wherein the anti-mycobacterial compound is isoniazid.

In certain embodiments, the implants described herein comprise an anti-mycobacterial compound, wherein the anti-mycobacterial compound is rifabutin.

In certain embodiments, the implants described herein comprise an anti-mycobacterial compound, wherein the anti-mycobacterial compound is rifapentine.

In certain embodiments, the implants described herein comprise a bulking agent, wherein the bulking agent is lactose.

In certain embodiments, the implants described herein further comprise a lubricant as a pharmaceutical excipient.

In certain embodiments, the implants described herein further comprise a lubricant as a pharmaceutical excipient, wherein the lubricant is magnesium stearate.

In certain embodiments, the implants described herein are suitable for sterilization, or have been sterilized, by irradiation.

In certain embodiments, the implants described herein comprise between 10 and 90% of the anti-mycobacterial compound by weight.

In certain embodiments, the implants described herein comprise between 50 and 90% of the anti-mycobacterial compound by weight.

In certain embodiments, the implants described herein comprise between 80% of the anti-mycobacterial compound by weight.

In certain embodiments, the implants described herein are rod-shaped. Preferably, the cross section of the implants described herein is circular.

Also described herein are processes for the production of the implants described herein. In certain embodiments, described herein is a process for the production of an implant comprising mixing an anti-mycobacterial compound with at least one pharmaceutical excipient and compressing the mixture into the desired shape. In certain embodiments, described herein is a process for the production of an implant comprising mixing an anti-tuberculosis compound with at least one pharmaceutical excipient and compressing the mixture into the desired shape.

Also described herein are 1) methods of treating tuberculosis in a subject in need of treatment thereof, comprising administering to the subject an implant described herein; and 2) uses of an implant described herein for the treatment of tuberculosis.

Also described herein are 1) methods of treating latent tuberculosis infection in a subject in need of treatment thereof, comprising administering to the subject an implant described herein; and 2) uses of an implant described herein for the treatment of latent tuberculosis.

Also described herein are 1) methods of treating non-tuberculous mycobacterial (NTM) disease in a subject in need of treatment thereof, comprising administering to the subject an implant described herein; and 2) uses of an implant described herein for the treatment of non-tuberculous Mycobacterial disease.

Also described herein are 1) methods of preventing tuberculosis in a subject at risk of becoming infected with tuberculosis, comprising administering to the subject an implant described herein; and 2) uses of an implant described herein for the prevention of tuberculosis.

Also described herein are 1) methods of preventing latent tuberculosis infection in a subject in need of treatment thereof, comprising administering to the subject an implant described herein; and 2) uses of an implant described herein for the treatment of latent tuberculosis.

Also described herein are 1) methods of preventing non-tuberculous mycobacterial (NTM) disease in a subject at risk of becoming infected with a non-tuberculous mycobacterial (NTM) disease, comprising administering to the subject an implant described herein; and 2) uses of an implant described herein for the prevention of non-tuberculous Mycobacterial disease.

Also described herein are methods for the treatment or prevention of tuberculosis infections comprising administering an implant described herein to an animal at risk of becoming infected or in need of such treatment. In certain embodiments of the methods described herein, the animal is a human. Various embodiments and features of the present invention are either further

described in or will be apparent from the ensuing description, examples and appended claims.

### FIGURES

The invention is illustrated by the following examples, and the accompanying figures in which:

FIG. 1 shows in vitro drug release kinetics of the implants prepared in Examples 1-4.

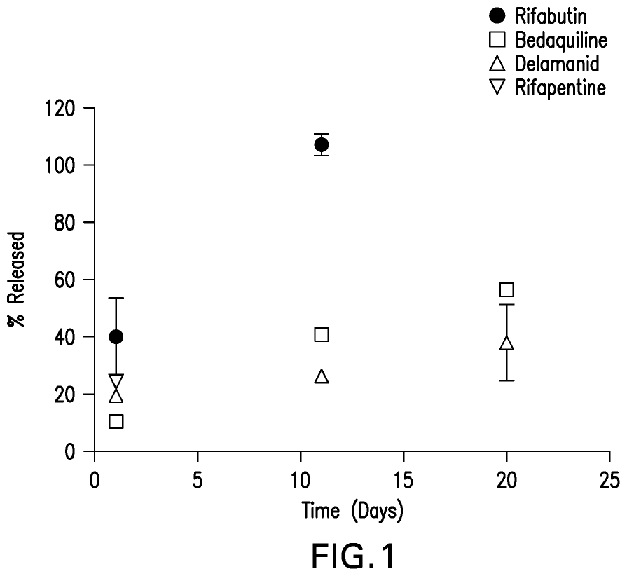

### DETAILED DESCRIPTION OF THE INVENTION

As used herein, “animal” “subject” and “patient” are used interchangeably and refers to humans (male or female), companion animals (e.g., dogs, cats and horses), food-source animals, zoo animals, marine animals, birds and other similar animal species. In specific embodiments herein, “animal” “subject” or “patient” refers to humans.

As used herein. “bulking agent” means a compaction aide.

Described herein are implants comprising at least one anti-mycobacterial compound and at least one pharmaceutical excipient, wherein at least one pharmaceutical excipient is a bulking agent.

Also described herein are implants comprising at least one anti-mycobacterial compound and pharmaceutical excipients, wherein at least one pharmaceutical excipient is a bulking agent.

Also described herein are implants comprising at least two anti-mycobacterial compounds and pharmaceutical excipients, wherein at least one pharmaceutical excipient is a bulking agent.

Also described herein are implants comprising at least two anti-mycobacterial compounds and at least one pharmaceutical excipient, wherein the at least one pharmaceutical excipient is a bulking agent.

Also, described herein are implants comprising at least one anti-mycobacterial compound and nonpolymer pharmaceutical excipients, wherein at least one nonpolymer pharmaceutical excipient is a bulking agent.

Also described herein are implants comprising at least one anti-mycobacterial compound and only nonpolymer pharmaceutical excipients, wherein at least one nonpolymer pharmaceutical excipient is a bulking agent.

Also described herein are implants comprising at least one anti-mycobacterial compound and at least one nonpolymer pharmaceutical excipient, wherein at least one nonpolymer pharmaceutical excipient is a bulking agent.

Also described herein are implants comprising at least two anti-mycobacterial compounds and nonpolymer pharmaceutical excipients, wherein at least one nonpolymer pharmaceutical excipient is a bulking agent.

Also described herein are implants comprising at least two anti-mycobacterial compounds and only nonpolymer pharmaceutical excipients, wherein at least one nonpolymer pharmaceutical excipient is a bulking agent.

Also described herein are implants comprising at least two anti-mycobacterial compounds and at least one nonpolymer pharmaceutical excipient, wherein the at least one nonpolymer pharmaceutical excipient is a bulking agent.

Also described herein are implants comprising at least one anti-tuberculosis compound and at least one pharmaceutical excipient, wherein at least one pharmaceutical excipient is a bulking agent.

Also described herein are implants comprising at least one anti-tuberculosis compound and pharmaceutical excipients, wherein at least one pharmaceutical excipient is a bulking agent.

Also described herein are implants comprising at least two anti-tuberculosis compounds and pharmaceutical excipients, wherein at least one pharmaceutical excipient is a bulking agent.

Also described herein are implants comprising at least two anti-tuberculosis compounds and at least one pharmaceutical excipient, wherein at least one pharmaceutical excipient is a bulking agent.

Also described herein are implants comprising at least one anti-tuberculosis compound and nonpolymer pharmaceutical excipients, wherein at least one nonpolymer pharmaceutical excipient is a bulking agent.

Also described herein are implants comprising at least one anti-tuberculosis compound and only nonpolymer pharmaceutical excipients, wherein at least one nonpolymer pharmaceutical excipient is a bulking agent.

Also described herein are implants comprising at least one anti-tuberculosis compound and at least one nonpolymer pharmaceutical excipient, wherein at least one nonpolymer pharmaceutical excipient is a bulking agent.

Also described herein are implants comprising at least two anti-tuberculosis compounds and nonpolymer pharmaceutical excipients, wherein at least one nonpolymer pharmaceutical excipient is a bulking agent.

Also described herein are implants comprising at least two anti-tuberculosis compounds and only nonpolymer pharmaceutical excipients, wherein at least one nonpolymer pharmaceutical excipient is a bulking agent.

Also described herein are solid implants comprising at least two anti-tuberculosis compounds and at least one nonpolymer pharmaceutical excipient, wherein at least one nonpolymer pharmaceutical excipient is a bulking agent.

In certain embodiments, suitable anti-tuberculosis compounds for the treatment of drug-susceptible TB, drug-resistant, TB or LTBI include, but are not limited to, bedaquiline, delamanid, linezolid, PBTZ169, pretomanid, Q203, rifampicin, rifapentine, isoniazid and sutezolid. Other suitable compounds include compounds described in PCT published application WO2016/064982.

In other embodiments, suitable anti-tuberculosis compounds are those having an aqueous solubility below 200 μg/ml in phosphate buffered saline (PBS), for example bedaquiline, delamanid, rifabutin and rifapentine. The table below (Table 1) shows the solubility of certain anti-tuberculosis compounds. In certain embodiments of the implants described herein, wherein the anti-tuberculosis compounds have low solubility, and wherein lactose is used as a bulking agent in the formulations, the lactose could also help to solubilize the drug and speed up the drug release.

TABLE 1
Solubility of four different anti-tuberculosis compounds

| Anti-tuberculosis | | |
|---|---|---|
| Compounds | Dissolution Media | Solubility at 37° C. |
| delamanid | PBS | 10.7 μg/ml |
| bedaquiline | PBS | 129 μg/ml |
| rifapentine | PBS | 134 μg/ml |
| rifabutin | PBS | 101 μg/ml |

In certain embodiments of the implants described herein, the implant contains one anti-tuberculosis compound, selected from the list comprising of bedaquiline, delamanid, linezolid, PBTZ169, pretomanid, Q203, rifampicin, rifapentine, isoniazid and sutezolid.

In certain embodiments of the implants described herein, the implant contains two anti-tuberculosis compounds, selected from the list comprising of bedaquiline, delamanid, linezolid, PBTZ169, pretomanid, Q203, rifampicin, rifapentine, isoniazid and sutezolid.

In certain embodiments of the implants described herein, the implant contains a combination of anti-tuberculosis compounds selected from the list comprising of bedaquiline, delamanid, linezolid, PBTZ169, pretomanid, Q203, rifampicin, rifapentine, isoniazid and sutezolid.

In certain embodiments, the anti-mycobacterial compound (or compounds) makes up between 10% and 90% of the implant by weight. In certain embodiments, the anti-mycobacterial compound (or compounds) makes up between 20% and 90% of the implant by weight. In certain embodiments, the anti-mycobacterial compound (or compounds) makes up between 30% and 90% of the implant by weight. In certain embodiments, the anti-mycobacterial compound (or compounds) makes up between 40% and 90% of the implant by weight. In certain embodiments, the anti-mycobacterial compound (or compounds) makes up between 50% and 90% of the implant by weight. In certain embodiments, the anti-mycobacterial compound (or compounds) makes up between 60% and 90% of the implant by weight. In certain embodiments, the anti-mycobacterial compound (or compounds) makes up between 70% and 90% of the implant by weight. In certain embodiments, the anti-mycobacterial compound (or compounds) makes up between 80% and 90% of the implant by weight. In certain embodiments, the anti-mycobacterial compound (or compounds) makes up 80%of the implant by weight.

In certain embodiments, the anti-tuberculosis compound (or compounds) makes up between 10% and 90% of the implant by weight. In certain embodiments, the anti-tuberculosis compound (or compounds) makes up between 20% and 90% of the implant by weight. In certain embodiments, the anti-tuberculosis compound (or compounds) makes up between 30% and 90% of the implant by weight. In certain embodiments, the anti-tuberculosis compound (or compounds) makes up between 40% and 90% of the implant by weight. In certain embodiments, the anti-tuberculosis compound (or compounds) makes up between 50% and 90% of the implant by weight. In certain embodiments, the anti-tuberculosis compound (or compounds) makes up between 60% and 90% of the implant by weight. In certain embodiments, the anti-tuberculosis compound (or compounds) makes up between 70% and 90% of the implant by weight. In certain embodiments, the anti-tuberculosis compound (or compounds) makes up between 80% and 90% of the implant by weight. In certain embodiments, the anti-tuberculosis compound (or compounds) makes up 80%of the implant by weight.

In certain embodiments described herein, greater than 95% by weight of the implant is made up of an anti-tuberculosis compound and nonpolymer pharmaceutical excipients, more preferably greater than 99% by weight. In certain embodiments described herein, 100% by weight of the implant is made up of an anti-tuberculosis compound and nonpolymer pharmaceutical excipients.

The implants described herein include nonpolymer pharmaceutical excipients, one of which is a bulking agent. In certain embodiments, the bulking agent will make up about 0.5%-30% of the implant by weight. In certain embodiments, the bulking agent will make up about 0.5%-25% of the implant by weight. In certain embodiments, the bulking agent will make up about 5%-25% of the implant by weight. In certain embodiments, the bulking agent will make up about 10%-25% of the implant by weight. In certain embodiments, the bulking agent will make up about 10%-20% of the implant by weight.

In certain embodiments, the bulking agent makes up about 10% of the implant by weight. In certain embodiments, the bulking agent makes up about 11% of the implant by weight. In certain embodiments, the bulking agent makes up about 12% of the implant by weight. In certain embodiments, the bulking agent makes up about 13% of the implant by weight. In certain embodiments, the bulking agent makes up about 14% of the implant by weight. In certain embodiments, the bulking agent makes up about 15% of the implant. In certain embodiments, the bulking agent makes up about 16% of the implant by weight. In certain embodiments, the bulking agent makes up about 17% of the implant by weight. In certain embodiments, the bulking agent makes up about 18% of the implant by weight. In certain embodiments, the bulking agent makes up about 19% of the implant by weight. In certain embodiments, the bulking agent makes up about 20% of the implant by weight.

Bulking agents include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. Preferred bulking agents include lactose or other sugars, microcrystalline cellulose (which is available commercially as AVICEL™) and dicalcium phosphate. A preferred group of carbohydrates includes lactose, threhalose, raffinose maltodextrins, and mannitol.

Suitable polypeptides include aspartame. Amino acids include alanine and glycine, with glycine being preferred.

Additional polymeric bulking agents include polyvinyl pyrrolidone (PVP), copovidone, crospovidone, polyvinyl alcohol (PVA), and the like.

In certain embodiments, the preferred bulking agent is lactose. In certain embodiments, lactose makes up about 0.5%-30% of the implant by weight. In certain embodiments, lactose makes up about 0.5%-25% of the implant by weight. In certain embodiments, lactose makes up about 5%-25% of the implant by weight. In certain embodiments, lactose makes up about 10%-25% of the implant by weight. In certain embodiments, lactose makes up about 10%-20% of the implant by weight.

In certain embodiments, lactose makes up about 10% of the implant by weight. In certain embodiments, lactose makes up about 11% of the implant by weight. In certain embodiments, lactose makes up about 12% of the implant by weight. In certain embodiments, lactose makes up about 13% of the implant by weight. In certain embodiments, lactose makes up about 14% of the implant by weight. In certain embodiments, lactose makes up about 15% of the implant by weight. In certain embodiments, lactose makes up about 16% of the implant by weight. In certain embodiments, lactose makes up about 17% of the implant by weight. In certain embodiments, lactose makes up about 18% of the implant by weight. In certain embodiments, lactose makes up about 19% of the implant by weight. In certain embodiments, lactose makes up about 20% of the implant by weight.

The implants described herein may include other pharmaceutical excipients including, but not limited to, binders, solubility enhancers, disintegrants, lubricants, glidants, stabilizers, reducing agents, non-ionic surfactants, humectants, antioxidants, fillers and diluents.

They also can impart desired characteristics to the finished product such as strength, solubility, bioavailability and the like, provided that these additional agents also are biocompatible, biodegradable and not anti-inflammatory.

Solubilizing agents include, but are not limited to, polyethylene glycol, Poloxamer 407, propylene glycol, hydroxypropyl-b-cyclodextrin, sulfobutylether-β-cyclodextrin, α-cyclodextrin, phospholipids, castor oil, hydrogenated castor oil, solutol, sorbitan monooleate, sucrose, dextrose anhydrous, dextrose monohydrate, and mannitol and the like.

Lubricants include, but are not limited to, magnesium stearate, calcium stearate, sodium stearate, talc, STEROTEX (food grade vegetable powders), waxes, STEAK-O-WET (spray-dried blend of magnesium stearate and sodium Lauryl Sulfate), glyceryl behapate, liquid paraffin and the like.

In certain embodiments of the implants described herein, a lubricant is present. Typically, the lubricant will make up about 0.5%-3.0% of the implant, by weight. In certain embodiments, the lubricant will make up about 0.5%-2.5% of the implant. In certain embodiments, the lubricant will make up about 0.5%-2.0% of the implant. In certain embodiments, the lubricant will make up about 0.5%-1.5% of the implant. In certain embodiments, the lubricant will make up about 0.5%-1.0% of the implant.

In certain embodiments, the lubricant will make up about 1.0% of the implant. In certain embodiments, the lubricant will make up about 2.0% of the implant.

Suitable lubricants include, but are not limited to, common minerals like talc or silica, and fats, e.g. vegetable stearin, magnesium stearate or stearic acid. In certain embodiment, magnesium stearate is included as a lubricant. In certain embodiments, magnesium stearate will make up about 0.5%-3.0% of the implant, by weight. In certain embodiments, magnesium stearate will make up about 0.5%-2.5% of the implant. In certain embodiments, magnesium stearate will make up about 0.5%-2.0% of the implant. In certain embodiments, magnesium stearate will make up about 0.5%-1.5% of the implant. In certain embodiments, magnesium stearate will make up about 0.5%-1.0% of the implant.

In certain embodiments, magnesium stearate will make up about 1.0% of the implant. In certain embodiments, magnesium stearate will make up about 2.0% of the implant.

Emulsifiers include, but are not limited to, glyceryl monostearate, stearic acid, stearyl alcohol,cetyl alcohol, and the like.

Humectants include, but are not limited to, glycerol, ethylene glycol, polyethylene glycol (PEG), diethylene glycol, triethylene glycol, propylene glycol, dipropylene glycol, glycerin, sorbitol, mannitol, glucose, and the like.

Nonionic surfactants include, but are not limited to, POE (20) sorbitan monooleate, also known as polyethylene glycol sorbitan monooleate, polyoxyethylenesorbitan monooleate, Polysorbate 80 and Tween 80® and the like.

Binding agents may also be included in the formulation to aid granulation and compressibility. Examples of binding agents include starch, gelatin and polyvinyl pyrrolidone. Typically, the binding agent, when present, will make up between 2 to 10% of the implant, by weight.

In certain embodiments, a further pharmaceutical excipient which the implants described herein may optionally contain is a disintegrant. Suitable disintegrants include sodium starch glycolate, which is available commercially as EXPLOTAB™. Other disintegrants which may be mentioned are dicalcium phosphate and cross-linked starch. Typically, the disintegrant, when present, will make up about 5% of the implant, by weight.

In certain embodiments, the implants described herein may contain an antioxidant or a reducing agent. It has been found that such additives reduce or eliminate degradation of the anti-tuberculosis compound, thus extending the shelf-life of the implant. It has been found that such additives are particularly useful for stabilizing the anti-tuberculosis compound when the implant is sterilized by irradiation, such as gamma or beta irradiation.

Suitable antioxidants include, but are not limited to, butylated hydroxy anisole (BHA; a mixture of 2-tert- butyl-4-methoxyphenol and 3-tert-butyl-4-methoxyphenol) and butylated hydroxy toluene (BHT; 2,6-di-tert-butyl-4-methylphenol). Other antioxidants and reducing agents include alpha-tocopherol, alkyl gallate derivatives, nordihydroguaiaretic acid, ascorbic acid, sodium metabisulphate and sodium sulphite. Typically, the antioxidant, when present, will make up between 0.01 to 0.5% of the implant, by weight, more preferably 0.1 to 0.2%.

Optionally, the implants described herein can further comprise a radio-opaque component. The radio-opaque component will cause the implant to be X-ray visible. The radio-opaque component can be any such element known in the art, such as barium sulphate, titanium dioxide, bismuth oxide, tantalum, tungsten or platinum. In a specific embodiment, the radio-opaque component is barium sulphate.

As mentioned above, the implants described herein may be terminally irradiated to sterilize them.

The size and shape of the implants described herein may be modified to achieve a desired overall dosage. In certain embodiments, the implants described herein are pellet-shaped. In certain embodiments, the implants described herein are tablet-shaped. In certain embodiments, the implants described herein are rod-shaped. In certain embodiments, the implants described herein have a circular cross-section.

In certain embodiments, the implants described herein can be about 0.5 cm to about 10 cm in length. In certain embodiments, the implants described herein are about 1.5 cm to about 5 cm in length. In certain embodiments, the implants described herein are about 2 cm to about 5 cm in length. In certain embodiments, the implants described herein are about 2 cm to about 4 cm in length.

In certain embodiments, the implants described herein can be about 0.5 mm to about 7 mm in diameter. In certain embodiments, the implants described herein are about 1.5 mm to about 5 mm in diameter. In certain embodiments, the implants described herein are about 2 mm to about 5 mm in diameter. In certain embodiments, the implants described herein are about 2 mm to about 4 mm in diameter.

Depending on the size of the implant and the dose of drug required to deliver a therapeutically effective amount of active ingredient, a therapeutically effective dose of the active ingredient can be delivered by implanting a single implant or multiple implants, e.g. 2-20 implants. More specifically, the number of implants implanted into a single subject or animal is 1, 2, 3, 4, 5, 6, 7, 8, 9 or 10 or more implants.

To facilitate implantation, the implants are preferably rod-shaped, and can be implanted conveniently using a conventional hand-operated implant applicator.

Implants according to the invention may be implanted intramuscularly, subcutaneously or subdermally. Preferably, however, they are implanted subdermally (i.e., directly below the skin). The implants of the invention may be implanted in various parts of the patient to be treated, for example the upper arm, gluteus maximus, thigh or abdomen. Preferably, however, they are implanted in the upper arm.

Implants described herein can be solid. In certain embodiments, depending on the pharmaceutical excipients used, the implants described herein can be bioabsorbable or biodegradable. In other embodiments, the implants described herein are not bioabsorbable or biodegradable.

According to the invention, there is also provided a method for the treatment or prevention of mycobacterial infections which comprises administering an implant as defined above to a subject in need of such treatment.

Also described herein are methods of treating tuberculosis in a subject in need of treatment thereof, comprising administering to the subject an implant described herein. Also described herein are uses of an implant described herein for the treatment of tuberculosis caused by infection with Mycobacterium tuberculosis, comprising administering to the subject an implant described herein.

Also described herein are methods of treating latent tuberculosis infection in a subject in need of treatment thereof, comprising administering to the subject an implant described herein. Also described herein are uses of an implant described herein for the treatment of latent tuberculosis comprising administering to the subject an implant described herein.

Also described herein are methods of treating non-tuberculous Mycobacterial (NTM) disease in a subject in need of treatment thereof, comprising administering to the subject an implant described herein. Also described herein are uses of an implant described herein for the treatment of non-tuberculous Mycobacterial disease comprising administering to the subject an implant described herein. Non-tuberculous Mycobacterial disease is a set of tuberculosis-like infections caused by relatives of Mycobacterium tuberculosis. NTM infections of the lung are the most common and largely caused by two mycobacterial species: Mycobacterium avium complex and Mycobacterium abscessus complex. Other NTM pathogenic species that could be treated using the implants described herein include: Mycobacterium chelonae, Mycobacterium kansassi, Mycobacterium fortuitum.

Also described herein are methods of treating leprosy, caused by Mycobacterium leprae, in a subject in need of treatment thereof, comprising administering to the subject an implant described herein. Also described herein are uses of an implant described herein for the treatment of leprosy caused by Mycobacterium leprae, comprising administering to the subject an implant described herein.

As used herein, the term “continually released” refers to the drug being released into plasma at continuous rates for extended periods of time. The implant drug delivery system of the instant invention generally exhibits linear release kinetics for the drug in vivo, sometimes after an initial burst.

The dosage to be administered will depend on the patient to be treated and the anti-tuberculosis compound being used. Typically, an implant described herein will achieve a daily therapeutic concentration, in blood serum, between 100 ng/ml to 5000ng/ml. In certain embodiments, an implant according to the invention having the preferred dimensions mentioned above will achieve a daily therapeutic concentration between 100 ng/ml to 200 ng/ml. In certain embodiments, an implant according to the invention having the preferred dimensions mentioned above will achieve a daily therapeutic concentration between 100 ng/ml to 300 ng/ml. In certain embodiments, an implant according to the invention having the preferred dimensions mentioned above will achieve a daily therapeutic concentration between 100 ng/ml to 400 ng/ml. In certain embodiments, an implant according to the invention having the preferred dimensions mentioned above will achieve a daily therapeutic concentration between 100 ng/ml to 500 ng/ml. In certain embodiments, an implant according to the invention having the preferred dimensions mentioned above will achieve a daily therapeutic concentration between 100 ng/ml to 600 ng/ml. In certain embodiments, an implant according to the invention having the preferred dimensions mentioned above will achieve a daily therapeutic concentration between 100 ng/ml to 700 ng/ml. In certain embodiments, an implant according to the invention having the preferred dimensions mentioned above will achieve a daily therapeutic concentration between 100 ng/ml to 800 ng/ml. In certain embodiments, an implant according to the invention having the preferred dimensions mentioned above will achieve a daily therapeutic concentration between 100 ng/ml to 900 ng/ml. In certain embodiments, an implant according to the invention having the preferred dimensions mentioned above will achieve a daily therapeutic concentration between 100 ng/ml to 1000 ng/ml.

In certain embodiments, an implant according to the invention having the preferred dimensions mentioned above will achieve a daily therapeutic concentration between 1000 ng/ml to 2000 ng/ml. In certain embodiments, an implant according to the invention having the preferred dimensions mentioned above will achieve a daily therapeutic concentration between 1000 ng/ml to 3000 ng/ml. In certain embodiments, an implant according to the invention having the preferred dimensions mentioned above will achieve a daily therapeutic concentration between 1000 ng/ml to 4000 ng/ml. In certain embodiments, an implant according to the invention having the preferred dimensions mentioned above will achieve a daily therapeutic concentration between 1000 ng/ml to 5000 ng/ml.

The dosage to be administered will depend on the patient to be treated and the anti-tuberculosis compound being used. Typically, an implant described herein will achieve a daily therapeutic concentration, in blood serum, between 1 μg/ml to 50 μg/ml. In certain embodiments, an implant according to the invention having the preferred dimensions mentioned above will achieve a daily therapeutic concentration between 1 μg/ml to 20 μg/ml. In certain embodiments, an implant according to the invention having the preferred dimensions mentioned above will achieve a daily therapeutic concentration between 1 μg/ml to 30 μg/ml. In certain embodiments, an implant according to the invention having the preferred dimensions mentioned above will achieve a daily therapeutic concentration between 1 μg/ml to 40 μg/ml. In certain embodiments, an implant according to the invention having the preferred dimensions mentioned above will achieve a daily therapeutic concentration between 1 μg/ml to 50 μg/ml. In certain embodiments, an implant according to the invention having the preferred dimensions mentioned above will achieve a daily therapeutic concentration between 1 μg/ml to 60 μg/ml. In certain embodiments, an implant according to the invention having the preferred dimensions mentioned above will achieve a daily therapeutic concentration between 1 μg/ml to 70 μg/ml. In certain embodiments, an implant according to the invention having the preferred dimensions mentioned above will achieve a daily therapeutic concentration between 1 μg/ml to 80 μg/ml. In certain embodiments, an implant according to the invention having the preferred dimensions mentioned above will achieve a daily therapeutic concentration between 1 μg/ml to 90 μg/ml. In certain embodiments, an implant according to the invention having the preferred dimensions mentioned above will achieve a daily therapeutic concentration between 1 μg/ml to 100 μg/ml.

In certain embodiments, an implant according to the invention having the preferred dimensions mentioned above will achieve a daily therapeutic concentration between 10 μg/ml to 20 μg/ml. In certain embodiments, an implant according to the invention having the preferred dimensions mentioned above will achieve a daily therapeutic concentration between 10 μg/ml to 30 μg/ml. In certain embodiments, an implant according to the invention having the preferred dimensions mentioned above will achieve a daily therapeutic concentration between 10 μg/ml to 40 μg/ml. In certain embodiments, an implant according to the invention having the preferred dimensions mentioned above will achieve a daily therapeutic concentration between 10 μg/ml to 50 μg/ml.

Below is a list (Table 2) of anti-tuberculosis compounds that can be used in the implants described herein and there daily therapeutic concentration.

TABLE 2
Anti-tuberculosis Compounds
and the corresponding therapeutic concentrations

| Anti-tuberculosis | | |
|---|---|---|
| Compounds | Therapeutic Concentrations | |
| Rifampicin | 1-10 | μg/ml |
| Isoniazid | 10 | μg/ml |
| Pyrazinamide | 10 | μg/ml |
| Ethambutol | 1-5 | μg/ml |
| Rifabutin | 100-500 | ng/ml |
| Rifapentine | 1-5 | μg/ml |
| Delamanid | 100-500 | ng/ml |
| Bedaquiline | 1-5 | μg/ml |

The implants described herein can provide sustained release of an anti-tuberculosis or anti-mycobacterial compound (or compounds) for one week to up to 3 years. In certain embodiments, the implants described herein can provide sustained release of an anti-tuberculosis or anti-mycobacterial compound (compounds) for one week. In certain embodiments, the implants described herein can provide sustained release of an anti-tuberculosis or anti-mycobacterial compound (compounds) for two weeks. In certain embodiments, the implants described herein can provide sustained release of an anti-tuberculosis or anti-mycobacterial compound (compounds) for three weeks. In certain embodiments, the implants described herein can provide sustained release of an anti-tuberculosis or anti-mycobacterial compound (compounds) for four weeks. In certain embodiments, the implants described herein can provide sustained release of an anti-tuberculosis or anti-mycobacterial compound (compounds) for five weeks. In certain embodiments, the implants described herein can provide sustained release of an anti-tuberculosis or anti-mycobacterial compound (compounds) for six weeks.

In certain embodiments, the implants described herein can provide sustained release of an anti-tuberculosis or anti-mycobacterial compound (compounds) for one month. In certain embodiments, the implants described herein can provide sustained release of an anti-tuberculosis or anti-mycobacterial compound (compounds) for two months. In certain embodiments, the implants described herein can provide sustained release of an anti-tuberculosis or anti-mycobacterial compound (compounds) for three months. In certain embodiments, the implants described herein can provide sustained release of an anti-tuberculosis or anti-mycobacterial compound (compounds) for four months. In certain embodiments, the implants described herein can provide sustained release of an anti-tuberculosis or anti-mycobacterial compound (compounds) for five months. In certain embodiments, the implants described herein can provide sustained release of an anti-tuberculosis or anti-mycobacterial compound (compounds) for six months. In certain embodiments, the implants described herein can provide sustained release of an anti-tuberculosis or anti-mycobacterial compound (compounds) for seven months. In certain embodiments, the implants described herein can provide sustained release of an anti-tuberculosis or anti-mycobacterial compound (compounds) for eight months. In certain embodiments, the implants described herein can provide sustained release of an anti-tuberculosis or anti-mycobacterial compound (compounds) for nine months. In certain embodiments, the implants described herein can provide sustained release of an anti-tuberculosis or anti-mycobacterial compound (compounds) for ten months. In certain embodiments, the implants described herein can provide sustained release of an anti-tuberculosis or anti-mycobacterial compound (compounds) for eleven months. In certain embodiments, the implants described herein can provide sustained release of an anti-tuberculosis or anti-mycobacterial compound (compounds) for twelve months.

In certain embodiments, the implants described herein can provide sustained release of an anti-tuberculosis or anti-mycobacterial compound (compounds) for one year. In certain embodiments, the implants described herein can provide sustained release of an anti-tuberculosis or anti-mycobacterial compound (compounds) for two years.

The implants described herein can be prepared using a compression technique or a molding technique. Implants can be formed by compressing or molding active ingredient powder such as micronized powder, optionally using solubility enhancers to enhance solubility, lubricants to enhance processing of the formed implants, emulsifiers, humectants and nonionic surfactants.

The implants of the invention may also be prepared by dry- or wet-mass granulation followed by milling and compression into the desired shape using conventional techniques.

For example, an implant consisting of rifabutin, lactose and magnesium stearate could be prepared by dry-mass granulation using the following steps:

1. Blend components except magnesium stearate

2. Sieve through a screen

3. Blend

4. Add half of magnesium stearate

5. Blend

6. Compress into slugs

7. Mill slugs to granules

8. Collect desired size fraction of granules

9. Blend

10. Add remaining magnesium stearate

11. Blend

12. Compress into rods

The steps for wet-mass granulation are similar. However, with wet-mass granulation some components are dissolved in a solvent and sprayed onto other components while they are blending.

Thus, according to a further aspect of the invention, there is provided a process for the production of an implant as defined above, which comprises mixing the anti-tuberculosis compound with the pharmaceutical excipients and forming into the desired shape.

### EXAMPLES

### Formulation 1

Drug powder was blended with lactose and magnesium stearate at the following ratio: 80wt % rifabutin, 18wt % lactose, and 2wt % magnesium stearate. The blend was granulated, and then compressed at 7 kN with 2 mm diameter, 7 tip, round concave tablet tooling. The resulting implants were approximately 25 mg each.

### Formulation 2

Drug powder was blended with lactose and magnesium stearate at the following ratio: 80wt % bedaquiline, 18wt % lactose, and 2wt % magnesium stearate. The blend was granulated, and then compressed at 7 kN with 2 mm diameter, 7 tip, round concave tablet tooling. The resulting implants were approximately 25 mg each.

### Formulation 3

Drug powder was blended with lactose and magnesium stearate at the following ratio: 80wt % delamanid, 18wt % lactose, and 2wt % magnesium stearate. The blend was granulated, and then compressed at 7 kN with 2 mm diameter, 7 tip, round concave tablet tooling. The resulting implants were approximately 25 mg each.

### In Vitro Drug Release of Formulations 1-3

The in vitro release rate of the drug was determined by incubating individual implants in a glass vial with phosphate buffered saline (PBS) containing 0.2 or 5wt % sodium dodecyl sulfate (SDS) at 37° C., and 50 rpm shaking in an Innova 42 incubator. The volume of media was sufficient to maintain sink conditions. Sink conditions are defined as the drug concentration maintained at or below ⅓ of the maximum solubility. Samples were removed at selected time points, and centrifuged at 20,800×g for 8 min. The supernatant was analyzed by HPLC. Results are shown in Table 3.

TABLE 3
Cumulative in vitro drug release from compressed implants
containing rifabutin, bedaquiline, and delamanid

| | rifabutin | | bedaquiline | | Delamanid | |
|---|---|---|---|---|---|---|
| | | Std. | | Std. | | Std. |
| Time | Avg. | Dev. | Avg. | Dev. | Avg. | Dev. |
| (days) | (%) | (%) | (%) | (%) | (%) | (%) |
| 1 | 40.6 | 13.4 | 10.9 | 0.8 | 20.3 | 1.3 |
| 11 | 107.7 | 3.6 | 41.4 | 2.9 | 26.8 | 1.1 |
| 20 | | | 57.2 | 1.3 | 38.3 | 13.2 |

[avg. = average and
std. dev. = standard deviation,
BLQ = below the limit of quantitation].

### In Vivo Drug Release of Examples 1-3

For each implantation, a Wistar Han rat was anesthetized using isoflurane to effect prior to subcutaneous dose administration. Using a trocar needle, three implants from each formulation example above were placed subcutaneously in the intrascapular region of a rat. Three animals were used for each formulation. Animals were monitored until recovered. At indicated time points, samples of blood were obtained from anesthetized animals (using isoflurane) and processed to plasma for determination of drug concentration by LC/MS. Results are shown in the below table (Table 4).

TABLE 4
Drug concentration in blood plasma from compressed implants
containing rifabutin, bedaquiline, and rifapentine

| | Rifabutin | | Bedaquiline | | Rifapentine | |
|---|---|---|---|---|---|---|
| | | Std. | | Std. | | Std. |
| Time | Avg. | Dev. | Avg. | Dev. | Avg. | Dev. |
| (days) | (nM) | (nM) | (nM) | (nM) | (nM) | (nM) |
| 0.083 | 81.9 | 19.9 | BLQ | BLQ | 630 | 409 |
| 0.167 | 164 | 34.8 | BLQ | BLQ | 803 | 367 |
| 0.25 | 244 | 47.3 | BLQ | BLQ | 1250 | 189 |
| 1 | 209 | 72.5 | BLQ | BLQ | 3530 | 539 |
| 2 | 182 | 44.5 | BLQ | BLQ | 5210 | 392 |
| 9 | 119 | 32.8 | 81 | 35.6 | 5980 | 606 |
| 16 | 75.9 | 38.5 | 63.6 | 39.7 | 2260 | 371 |
| 30 | 69.3 | 24.4 | 51.7 | 31.7 | 1010 | 269 |
| 44 | 42.3 | 3.39 | 70.3 | 28.6 | 1460 | 1040 |
| 58 | 38.3 | 14.1 | 60.2 | 27.1 | 477 | 541 |

[avg. = average and
std. dev. = standard deviation,
BLQ = below the limit of quantitation].

What is claimed:

1. An implant comprising at least one anti-mycobacterial compound; and nonpolymer pharmaceutical excipients, wherein at least one nonpolymer pharmaceutical excipient is a bulking agent.

2. The implant of claim 1, wherein the implant is adapted for subdermal implantation.

3. The implant of claim 1, wherein the anti-mycobacterial compound is an anti-tuberculosis compound.

4. The implant of claim 1, wherein the anti-mycobacterial compound has low aqueous solubility.

5. The implant of claim 1, wherein the anti-mycobacterial compound has an aqueous solubility below 200 μg/ml.

6. The implant of claim 1, wherein the anti-mycobacterial compound is bedaquiline, delamanid, linezolid, PBTZ169, pretomanid, Q203, rifampicin, rifabutin, rifapentine, isoniazid or sutezolid.

7. The implant of claim 1, wherein the anti-mycobacterial compound is bedaquiline.

8. The implant of claim 1, wherein the anti-mycobacterial compound is rifabutin.

9. The implant of claim 1, wherein the anti-mycobacterial compound is rifapentine.

10. The implant of claim 1, wherein the bulking agent is lactose.

11. The implant of claim 1, further comprising a lubricant as a pharmaceutical excipient.

12. The implant of claim 1, wherein the lubricant is magnesium stearate.

13. The implant of claim 1, wherein the implant is suitable for sterilization, or has been sterilized, by irradiation.

14. The implant of claim 1, wherein the implant comprises between 10 and 90% of the anti-mycobacterial compound by weight.

15. The implant of claim 1, wherein the implant comprises between 50 and 90% of the anti-mycobacterial compound by weight.

16. The implant of claim 1, wherein the implant comprises 80% of the anti-mycobacterial compound by weight.

17. The implant of claim 1, wherein the implant is rod-shaped.

18. A process for the production of an implant of claim 1, comprising mixing the anti-tuberculosis compound with at least one pharmaceutical excipient and compressing the mixture into the desired shape.

19. A method for the treatment or prevention of tuberculosis infections comprising administering an implant of claim 1 to an animal in need of such treatment.

20. The method of claim 19, wherein the animal is a human.

21. An implant comprising at least one anti-mycobacterial compound and pharmaceutical excipients, wherein at least one pharmaceutical excipient is a bulking agent.

22. The implant of claim 21, wherein the anti-mycobacterial compound is bedaquiline, delamanid, linezolid, PBTZ169, pretomanid, Q203, rifampicin, rifabutin, rifapentine, isoniazid or sutezolid.

23. The implant of claim 21, wherein the anti-mycobacterial compound is bedaquiline.

24. The implant of claim 21, wherein the anti-mycobacterial compound is rifabutin.

25. The implant of claim 21, wherein the anti-mycobacterial compound is rifapentine.

26. The implant of claim 21, wherein the anti-mycobacterial compound is isoniazid.

27. The implant of claim 21, wherein the bulking agent is lactose.
